(19) United States
(12) Patent Application Publication
(10) Pub. No.: US 2003/0097104 A1
(43) Pub. Date: May 22, 2003

(54) Tampon with a blended non-woven overwrap

(75) Inventor: Steven Ray Gilbert, Fairfield, OH (US)

Correspondence Address: THE PROCTER & GAMBLE COMPANY, INTELLECTUAL PROPERTY DIVISION, WINTON HILL TECHNICAL CENTER - BOX 161, 6110 CENTER HILL AVENUE, CINCINNATI, OH, 45224, US

(73) Assignee: The Procter & Gamble Company

(21) Appl. No.: 10/298,890

(22) Filed: Nov. 18, 2002

Related U.S. Application Data
(60) Provisional application No. 60/333,092, filed on Nov. 16, 2001.

(51) Int. Cl. A61F 13/20

(52) U.S. Cl. 604/370; 604/385.17; 604/904

(57) ABSTRACT

An improved absorbent catamenial tampon having a non-aggressive fluid wicking overwrap, which aids in fluid acquisition while not aggressively adhering to tissue is disclosed. The tampon comprises a generally elongated absorbent member and an overwrap substantially covering the exterior surface of the generally elongated absorbent member, wherein the overwrap comprises a fibrous non-woven material;

the fibrous non-woven material comprising a combination of synthetic fibers and rayon fibers; wherein the ratio of the synthetic fibers to rayon fibers is from about 90:10 to about 30:70.

### CROSS REFERENCE TO RELATED APPLICATIONS

This application claims the benefit of U.S. Provisional Application No. 60/333,092, filed Nov. 16, 2001.

### FIELD OF THE INVENTION

This invention relates to absorbent tampons having an overwrap that does not aggressively adhere to tissue, yet provides high levels of fluid wicking ability and improved absorbent characteristics. The non-aggressive, fluid wicking overwrap substantially covers the exterior surface of the generally elongated absorbent member.

### BACKGROUND OF THE INVENTION

A wide variety of absorbent catamenial tampons have long been known in the art. The exterior surface of tampons affects the fluid acquisition rate and thus the absorbency and expansion properties. The surface also affects the comfort of the tampon for the user. Often these two important functions of the exterior surface of the tampon are in conflict with one another. Materials which aid in fluid acquisition due to their strong affinity for fluid, typically also have a strong affinity for moist tissue and therefore adhere to the tissue of the user. Thus, many tampons trade off one function while trying to maximize the other. For example, overwraps designed mainly with fluid acquisition in mind have included the use of 100% hydrophilic fibers or the use of hydrophilic finishes on initially hydrophobic fibers. On the other hand, overwraps and/or exterior surfaces of tampons designed mainly with comfort in mind have included the use of hydrophobic materials and/or treatments. Thus, there is a need for materials for use in the exterior surface of tampons that have a strong affinity for fluid yet do not adhere aggressively to tissue. The superior design of the present invention will achieve better absorbency without adhering aggressively to vaginal tissue.

### BACKGROUND ART

An International Patent application WO 99/00096 published by Gell et al. on Jan. 7, 1999 relates to a tampon having an apertured film cover.

### SUMMARY OF THE INVENTION

This invention relates to improved tampons comprising a generally elongated absorbent member and a non-aggressive, fluid wicking overwrap substantially covering the exterior surface of the absorbent member.

### BRIEF DESCRIPTION OF THE DRAWINGS

While the specification concludes with claims particularly pointing out and distinctly claiming the subject matter which is regarded as forming the present invention, it is believed that the invention will be better understood from the following description taken in conjunction with the accompanying drawing, in which:

FIG. 1 is a perspective view of a tampon of the present invention incorporating a generally elongated absorbent member and non-aggressive, fluid wicking overwrap covering the exterior surface.

### DETAILED DESCRIPTION OF THE INVENTION

The present invention utilizes a non-aggressive, fluid wicking overwrap that covers the exterior surface of the generally elongated absorbent member to provide a comfortable insertion and withdrawal to the user. It also delivers a high level of fluid wicking ability, important to the absorptive performance of the product and reduction in soiling for the consumer. This non-aggressive, fluid wicking overwrap comprises a combination of synthetic fibers and rayon fibers. The ratio of the synthetic fibers to rayon fibers is from about 90:10 to about 30:70.

As used herein the term “tampon” refers to any type of absorbent structure that is inserted into the vaginal canal or other body cavities for the absorption of fluid therefrom. Typically, tampons are constructed from a generally elongated absorbent member that has been compressed and/or formed into a vaginally insertable shape.

As used herein the terms “vaginal cavity,” “within the vagina” and “vaginal interior,” are intended to be synonymous and refer to the internal genitalia of the human female in the pudendal region of the body. The term “vaginal cavity” as used herein is intended to refer to the space located between the introitus of the vagina (sometimes referred to as the sphincter of the vagina) and the cervix and is not intended to include the interlabial space, including the floor of vestibule. The externally visible genitalia generally is not included within the term “vaginal cavity” as used herein.

As used herein “adhesion to tissue” refers to the undesired union of the exterior surface of the tampon with the internal surface of the vagina which is believed to be caused by both mechanical and capillary action. The capillary action refers to the tendency of the exterior surface of a tampon to attract or suction itself to the walls of the vaginal cavity until it is satisfied by fluid due to the capillary strength of the exterior surface. Mechanical action is related to the level of surface contact and the roughness of the substrate. Such adhesion is typically associated with an uncomfortable, or even painful, sensation when the tampon is inserted and/or withdrawn from the vaginal cavity due to tugging or pulling of the rugae (or folds) of the vaginal wall.

As herein “aggressive” or “aggressiveness” refers to a materials tendency to cause adhesion to tissue. Aggressive materials are typically composed of high capillary strength natural fibers, such as rayon, which wick fluid readily and adhere to tissue. Alternatively “Non-aggressive” as used herein refers to materials which have a lower tendency to adhere to tissue which typically include synthetic fibers.

As used herein “fluid wicking” refers to the ability of a medium to carry fluid or moisture by capillary action. The fluid wicking capacity of a medium can be measured by grams of fluid absorbed per gram of overwrap material at saturation. A method for quantifying such capacity is provided in the Test Method section below.

As used herein “compressed” refers to pressing or squeezing together or otherwise manipulating the size, shape, and/or volume to obtain a generally elongated absorbent member having vaginally insertable shape.

As used herein, “vaginally insertable shape” refers to the geometrical form of the absorbent tampon after compression. The tampon may be compressed into a generally cylindrical configuration in the radial direction along the longitudinal and/or lateral axes, axially, or in both the radial and axial directions. An example of a typical compressed tampon is one which is about 10-16 mm wide and about 40-50 mm long depending on absorbency. While the tampon may be compressed into a substantially cylindrical configuration, other shapes are possible. These may include shapes having a cross section that may be described as rectangular, triangular, trapezoidal, semi-circular, hourglass, or other suitable shapes.

Unless specifically stated otherwise, as used herein a first material is “substantially covering” or “substantially covers” a second material when the first material covers at least about 75%, typically at least about 90% of the surface area of the second material.

The term “joined” or “attached” as used herein, encompasses configurations in which a first element is directly secured to second element by affixing the first element directly to the second element; configurations in which the first element is indirectly secured to the second element by affixing the first element to intermediate member(s) which in turn are affixed to the second element; and configurations in which first element is integral with second element; i.e., first element is essentially part of the second element.

As used herein, “cm” is centimeter, “mm” is millimeters, “g” is grams, “gsm” is grams per meter squared, “dpf”is denier per fiber, “g/g”is gram of fluid per gram of sample, “wt” is weight, “psi” is pound per square inch..

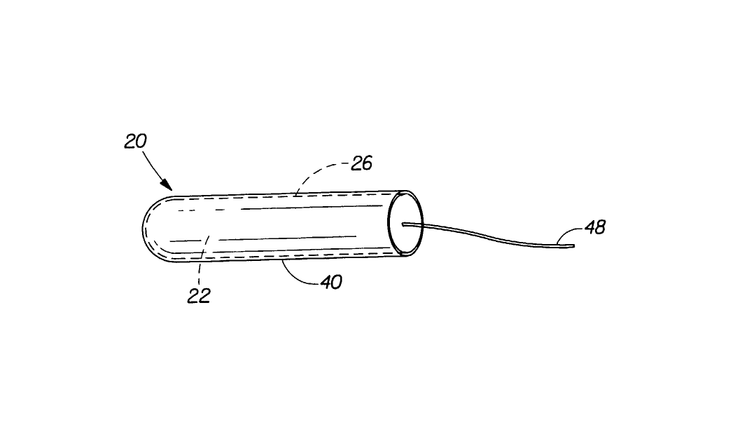

FIG. 1 shows one embodiment of such an absorbent tampon 20. The present invention, however, is not limited to a structure having the particular configuration shown in the drawing. The generally elongated absorbent member 22 (sometimes also referred to as the “absorbent core”) of the tampon 20 is shown in FIG. 1. The generally elongated absorbent member has an exterior surface 26. To form a tampon ready for use, the generally elongated absorbent member 22 is typically compressed and optionally heat conditioned in any suitable conventional manner. The exterior surface 26 of the generally elongated absorbent member 22 is substantially covered by the non-aggressive, fluid wicking overwrap 40. In one embodiment, the tampon 20 includes a withdrawal means 48 as described below in more detail.

I. Tampon of the Present Invention:

The tampon of the present invention comprises a generally elongated absorbent member and non-aggressive, fluid wicking overwrap that substantially covers the exterior surface of the generally elongated absorbent member.

a. Generally Elongated Absorbent Member

The generally elongated absorbent member 22 comprises absorbent material which is compressed or formed into a vaginally insertable shape. The absorbent material may be generally square or rectangular or take on other shapes such as trapezoidal, triangular, hemispherical, chevron or hourglass shapes. A typical size for absorbent material prior to compression may be from about 40 mm to about 100 mm in length and from about 40 mm to about 80 mm in width. In general, the absorbent material may be from about 40 mm to about 60 mm in length and from about 50 mm to about 70 mm in width. The typical range for the overall basis weight is from about 150 gsm to about 800 gsm.

The absorbent material may be a laminar structure comprised of integral or discrete layers. In other embodiments, the pad need not have a layered structure at all. The absorbent material may comprise a folded structure or may be rolled. The resulting absorbent member 22 of the tampon 20 may be constructed from a wide variety of liquid-absorbing materials commonly used in absorbent articles such as rayon (including tri-lobal and conventional rayon fibers), cotton, or comminuted wood pulp which is generally referred to as airfelt. Examples of other suitable absorbent materials include creped cellulose wadding; meltblown polymers including coform; chemically stiffened, modified or cross-linked cellulosic fibers; synthetic fibers such as crimped polyester fibers; peat moss; foam; tissue including tissue wraps and tissue laminates; or any equivalent material or combinations of materials, or mixtures of these.

Typical absorbent materials comprise cotton, rayon folded tissues, woven materials, non-woven webs, synthetic and/or natural fibers or sheeting. The tampon and any component thereof may comprise a single material or a combination of materials. Additionally, superabsorbent materials, such as super absorbent polymers or absorbent gelling and open-celled foams, materials may be incorporated into the tampon.

The materials for the tampon can be formed into a fabric, web, or batt that is suitable for use in the absorbent material by any suitable process such as airlaying, carding, wetlaying, hydroentangling, needling or other known techniques.

In another non-limiting embodiment, the absorbent material and resulting absorbent member comprise rayon, cotton, or combinations of both materials. These materials have a proven record of suitability for use in the human body. The rayon used in the absorbent material may be any suitable type typically used in disposable absorbent articles intended for in vivo use. Such acceptable types of rayon include GALAXY Rayon (a tri-lobed rayon structure) available as 6140 Rayon from Acordis Fibers Ltd., of Hollywall, England. SARILLE L rayon (a round fiber rayon), also available from Acordis Fibers Ltd. is also suitable. Any suitable cotton material may be used in the generally elongated absorbent member. Suitable cotton material includes, long fiber cotton, short fiber cotton, cotton linters, T-fiber cotton, card strips, and comber cotton. Preferably, the cotton layers should be scoured and bleached cotton absorbent with a glycerin finish, or other suitable finish.

If the generally elongated absorbent member of the present invention is layered, the layers may comprise different materials. For example, in one embodiment, the outer layers may comprise primarily rayon, while the intermediate layer or layers may comprise primarily cotton. Optionally, the entire generally elongated absorbent member may comprise a uniform or non-uniform blend of materials throughout. In one layered embodiment, each of the layers may comprise essentially 100% of the same material, such as outer layers of 100% rayon and an intermediate layer of 100% cotton. A Super Plus absorbency tampon of the present invention may be made from a pledget comprising about 100% rayon fibers. A Super absorbency or regular absorbency tampon of the present invention may be made from a pledget comprising about 25% cotton and about 75% rayon fibers. A Junior absorbency tampon may be made from a pledget comprising about 50% cotton and about 50% rayon fibers.

Pressures and temperatures suitable for compression are well known in the art. While a variety of techniques are known and acceptable for these purposes, a modified tampon compressor machine available from Hauni Machines, Richmond, Va., is suitable.

b. Non-Aggressive, Fluid Wicking Overwrap:

The non-aggressive, fluid wicking overwrap (referred to below simply as “the overwrap”) refers to the liquid pervious material covering the exterior surface of the generally elongated absorbent member. The overwrap comprises a fibrous non-woven material, which comprises a combination of synthetic fibers and rayon fibers. The ratio of synthetic fibers to rayon fibers is from about 90:10 to about 30:70. The synthetic fibers include but are not limited to fibers such as polyester, polyolefin, nylon, polypropylene, polyethylene, polyacrylic, cellulose acetate or bicomponent fibers. In general, the rayon fibers provide ready absorption, while the synthetic fibers balance the capillary strength of the material, enabling the tampon to more readily slip against moist tissue, resulting in easier insertion and removal, hence removal comfort. Optionally, other fibers may also be incorporated, such as cotton fibers. In another embodiment, the ratio of synthetic fibers to rayon fibers fall in the range of from about 80:20 to about 25:75 or even from about 70:30 to about 40:60.

The synthetic fibers may have hydrophobic and hydrophilic finishes. The synthetic fibers may be inherently hydrophilic, or may be treated to provide such properties. The combination of fibers may be formulated with some level of inherently hydrophobic fiber or hydrophobically treated fiber as well, as long as it does not significantly diminish the fluid wicking strength of the overwrap.

The non-woven overwraps of the present invention may be mechanically altered in one or more directions in order to reduce the aggressiveness of the material. Any known means of mechanically altering films or non-wovens can be used in developing overwraps useful in the present invention. Mechanically altering thus includes the well known processes such as ring rolling or “pre-corrugating”, SELFing, and/or aperturing. One method of SELFing is disclosed in U.S. Pat. No. 5,518,801 to Chappell. Known methods of apperaturing include hot pin (e.g. U.S. Pat. No. 5,188,625 to Van Iten et al.), slit and stretch (e.g. U.S. Pat. No. 5,714,107 to Levy) and selectively aperaturing (U.S. Pat. No. 5,916,661 to Benson et al.). The reduction in surface area of contact, reduced capillary strengths (due to larger pores after stretching/aperturing) ,and also the elastic nature of the resulting fabrics , can individually and combine to reduce the shear force from vaginal tissue, and improve removal comfort (and Agar shear values) while still maintaining good fluid handling. Mechanically altering materials which normally have very high agar shear values enables the use of a wider variety of materials having good wicking abilities since mechanically altering can lower the agar shear value to the desirable range.

The material can be made via any number of techniques. Commonly, carded webs that are hydroentangled, thermally bonded, needled, and resin bonded have application. The blending and layering of the synthetic and rayon fibers is well known in the art. In the case of resin bonded materials, the resin bonding agent can be used for tempering the aggressiveness of the rayon fiber matrix (10-30% bonding agent is common). A binder that reduces the adhesion to tissue, yet doesn't unacceptably degrade the wicking performance would be acceptable. The binder could be of a wide variety to include but not be limited to acrylates, acetates, styrene-isoprene, styrene-butadiene, polyvinylalchohols modified starches and the like.

Another technique to create the overwrap would utilize spunbond and meltblown processes. This technique would create a layer of less aggressive synthetic fibers that would be layered onto and intermeshed with a carded web of natural fibers.

The basis weight of the overwrap may be at least about 10 grams per square meter, optionally from about 10 to about 60 grams per square meter, alternatively from about 15 to about 30 grams per square meter.

In another embodiment includes a material that is dual layered with an outside and inside layer, made in accordance with U.S. Pat. No. 5,273,596. In this case, the outside layer is a 75% hydrophilically treated polypropylene with a 2.2 dpf and 25% 1.5 dpf rayon. The inside layer is 25% hydrophilically treated polypropylene with a 2.2 dpf and 75% 1.5 dpf rayon. The basis weights of the layers can vary, having from about 10 to about 15 grams per square meter in each layer. The resultant material is a 50% rayon 50% polypropylene thermally bonded blend with a basis weight from about 20 to about 30 grams per square meter.

The overwrap possesses a horizontal wicking capacity (described in the test method below) greater than about 1, alternatively greater than about 3 grams of fluid per gram of overwrap. Yet another embodiment includes overwraps having a horizontal wicking capacity of from about 6 to about 10 grams of fluid per gram of overwrap.

In the embodiment shown, the overwrap 40 material is generally rectangular, but other shapes such as trapezoidal, triangular, hemispherical, chevron, hourglass shaped, “T” and “L” shaped are also acceptable. Optimally, the overwrap may correspond to the shape of the generally elongated absorbent member. The overwrap positioned around the absorbent member so that the overwrap may be proximate with the insertion end and the withdrawal end of the generally elongated absorbent member. In this regard, the overwrap could exactly match up to the insertion end or withdrawal end could extend for example, 2 mm to 8 mm over either end. As well, the overwrap may extend over the withdrawal end further to form an optional skirt portion as discussed below.

In addition to substantially covering the elongated absorbent member, the overwrap may substantially cover both the first surface and the second surface of the absorbent material prior to compression. “Substantially covers” in this case means that the overwrap covers at least about 50%, optionally at least about 75% or even 90% of the combined surface area of the first surface and the second surface. Thus, for example, the overwrap “substantially covers” the first surface and the second surface of the absorbent material when it covers 100% of the first surface and 50% of the second surface. The overwrap may be wrapped around the longitudinal axis “L” or the transverse axis “T” as shown in the attached figures in another embodiment. As well, two separate pieces of overwrap can sandwich the absorbent material.

The overwrap may be joined to the generally elongated absorbent member by any variety of means. The overwrap may be joined to itself or to the generally elongated absorbent member. For example, one portion of the overwrap may be joined to an opposed portion of the overwrap or the absorbent member using any suitable adhesive or heat/pressure bonding means. Such adhesive may extend continuously along the length of attachment or it may be applied in a “dotted” fashion at discrete intervals. One method of heat bonding includes thermally bonding, fusion bonding, or any other suitable means known in the art for joining such materials. Alternatively, the overwrap may be joined to the generally elongated absorbent member along with the withdrawal cord by stitching. Such stitching may use cotton or rayon thread.

c. Optional Components

In one embodiment, the tampon of the present invention may comprise a withdrawal means. The withdrawal means, may be joined to the tampon for removal of the tampon after use. The withdrawal means may be joined to at least the primary generally elongated absorbent member and extends beyond at least the withdrawal end. Any of the withdrawal means currently known in the art may be used as a suitable withdrawal mechanism. In addition, the withdrawal means can take on other forms such as a ribbon, loop, tab, or the like. The withdrawal means may be integral with the generally elongated absorbent member.

The withdrawal means may be non-absorbent along at least the location of attachment to the generally elongated absorbent member. As used herein, the term “non-absorbent” refers to a structure that does not retain a significant portion of deposited fluid in its structure. The entire withdrawal means or other withdrawal mechanism may be made non-absorbent, if desired. The materials comprising the withdrawal cord may be inherently non-wettable or hydrophobic, or they may be treated to provide such properties. For example, a coating of wax may be applied to the withdrawal cord to decrease or eliminate its absorbency. The withdrawal means need not necessarily be non-wicking, even if a non-absorbent withdrawal cord is desired. For example, it may be desirable to provide a withdrawal means in which at least a portion of the cord has a tendency to wick deposited fluid upwardly toward the withdrawal end of the generally elongated absorbent member and into the structure thereof.

The withdrawal means may be attached in any suitable manner known in the art including sewing, adhesive attachment, or a combination of known bonding methods. The withdrawal means may be joined to any suitable location on the tampon.

The tampon of the present invention may be inserted digitally or through the use of an applicator. Any of the currently available tampon applicators may also be used for insertion of the tampon of the present invention. Such applicators of typically a “tube and plunger” type arrangement and may be plastic, paper, or other suitable material. Additionally, a “compact” type applicator is also suitable.

The non-aggressive, fluid wicking overwrap may extend below the withdrawal end to form a skirt portion. The non-aggressive, fluid wicking overwrap can extend 1 mm, optionally 2 mm to 30 mm from the withdrawal end of the generally elongated absorbent member. In another embodiment, the non-aggressive, fluid wicking overwrap extends from about 5 mm to 20 mm from the withdrawal end of the generally elongated absorbent member. When this approach is used, it is desirable for the skirt portion to be substantially free of the generally elongated absorbent member. In one embodiment, the skirt is less dense than the generally elongated absorbent member.

Both the generally elongated absorbent member and skirt portion of the non-aggressive, fluid wicking overwrap may reside entirely within the vaginal cavity of the wearer during use of the tampon. This is achieved by the relatively closeness of the skirt portion to the withdrawal end of the generally elongated absorbent member as well of the relative size compared to the overall size of the tampon. In particularly preferred embodiments, only the withdrawal cord or other withdrawal mechanism reside externally to the orifice of the vagina.

II. Test Methods:

a. Horizontal Gravimetric Wicking Capacity

The horizontal gravimetric wicking capacity is a measure of a materials ability to absorb fluid by capillary action. The horizontal gravimetric wicking capacity is measured by the Horizontal Gravimetric Wicking Test (HGW). This is an absorbency test that measures the uptake of fluid by a single layer of the overwrap fabric. This test is run at a controlled temperature of 73° F.±4° F. (22.8° C.±−15.6° C.) and relative humidity of 50%±4%. Overwrap samples should be conditioned at this temperature and humidity level for about 24 hours prior to running the test. In this method, a 2.25″ circular piece of material is cut and put in the sample holder, suspended from an electronic balance. The tampon is constrained under 0.06 psi (414 Pascals) pressure by a conformable member under air pressure which keeps the pressure relatively constant over the entire sample. A plastic supply tube, containing the test fluid (in this case, artificial menstrual fluid) is connected to a fluid reservoir at zero hydrostatic head relative to the test sample. A meniscus of fluid is brought in contact with a center point of the sample. The increase in weight of the sample is used as a measure of fluid up-take when the sample is saturated. Thus, the test is run until the sample reaches saturation (less than 0.01 g change over 30 seconds). The final sample weight is measured by removing the sample from the holder, and weighing it on an electronic balance. The g/g absorbency is then calculated as (final wt−dry wt)/dry weight. Three duplicate samples of each embodiment should be run and the average g/g absorbency used as the horizontal gravimetric wicking capacity of that embodiment.

b. Preparation of Artifical Menstrual Fluid:

Step 1: Dilute 2.5 ml of reagent grade 85-95% lactic acid to 27.5 ml with distilled water. Label as 8% lactic acid.

Step 2: Mix 10.0 g of KOH with 90 ml distilled water until completely dissolved. Label as 10% potassium hydroxide solution.

Step 3: Add 8.5 g sodium chloride and 1.38 g hydrous monobasic sodium phosphate to a flask and dilute to 100 ml with distilled water. Mix until completely dissolved. Label as monobasic sodium phosphate solution.

Step 4: Add 8.5 g sodium chloride and 1.42 g anhydrous dibasic sodium phosphate to flask and dilute to 100 ml with distilled water. Mix until completely dissolved. Label as dibasic sodium phosphate solution.

Step 5: Add 450 ml of the dibasic sodium phosphate solution to a 100 ml beaker and add monobasic sodium phosphate solution until the pH is lowered to 7.2±0.1. Label as phosphate solution.

Step 6: Mix 460 ml pf phosphate solution and 7.5 ml of 10% potassium hydroxide solution in a 100 ml beaker. Heat solution to 50° C. and then add 31 g sterilized gastric mucin (American Laboratories, Inc. Omaha Nebr.). Continue heating for 2.5 hours to completely dissolve the gastric mucin. Allow the solution to cool to less than 40° C. and then add 2.0 ml of 8% lactic acid solution. Autoclave mixture at 121° C. for 15 minutes, then allow to cool to room temperature. Mucin mixture should be used within & days>label as gastric mucin solution.

Step 7: Mix 500 ml of gastric mucin solution and 500 ml of fresh, sterile defibrinated sheep blood (Cleveland Scientific, American Biomedical, Bath, Ohio) in a beaker. The sheep blood should have a packed cell volume of greater than 38. The resulting artificial menstrual fluid should have a viscosity at 23° C. of between 7.15 and 8.64 centistokes. Label as artificial menstrual fluid. Store refrigerated and use within 7 days.

The following examples demonstrate particular embodiments of the present invention, but should not be construed to limit the scope of the invention.

### EXAMPLE 1

A tampon of the present invention was prepared. The tampon comprises an absorbent material of 75% rayon and 25% cotton fiber with a basis weight of 750 gsm having dimensions of about 70 mm in width and about 48 mm in length. The tampon also comprises an overwrap of hydroentangled 50% rayon and 50% polyester fibers with a basis weight of 37 gsm having dimensions of about 168 mm in width and about 48 mm in length. The overwrap is wrapped around the absorbent material such the overwrap material substantially covers the broad faces of the absorbent material. The absorbent material and overwrap are compressed axially and longitudinally, folding while being compressed, and then heated to form a generally elongated member substantially covered by the overwrap. The dimensions of the final tampon are approximately 14 mm diameter and approximately 46 mm length.

### EXAMPLE 2

A tampon of the present invention was prepared comprising an absorbent material of 75% rayon and 25% cotton fiber with a basis weight of 500 gsm having dimensions of about 100 mm in width and about 48 mm in length. The tampon also comprises an overwrap of hydroentangled 50% rayon and 50% polyester fibers with a basis weight of 37 gsm having dimensions of about 100 mm in width and about 48 mm in length. The overwrap is placed such that it covers one side of the absorbent material. The absorbent material and overwrap are rolled together so that the overwrap forms the outside of the tampon, then compressed axially and longitudinally and heated to form a generally elongated member substantially covered by the overwrap. The dimensions of the resulting tampon are approximately 14 mm diameter and approximately 46 mm length.

While particular embodiments of the present invention have been illustrated and described, it would be obvious to those skilled in the art that various other changes and modifications can be made without departing from the spirit and scope of the invention.

What is claimed is:

1. A catamenial tampon comprising:
a generally elongated absorbent member and an overwrap which substantially covers the absorbent member;
the overwrap comprising a fibrous non-woven material;
the fibrous non-woven material comprising a combination of synthetic fibers and rayon fibers;
wherein the ratio of the synthetic fibers to rayon fibers is from about 90:10 to about 30:70.

2. A tampon according to claim 1 wherein the ratio of the synthetic fibers to rayon fibers is from about 80:20 to about 25:75

3. A tampon according to claim 1 wherein the ratio of the synthetic fibers to rayon fibers is from about 70:30 to about 40:60.

4. A tampon according to claim 1 wherein the synthetic fibers comprise a hydrophilic finish.

5. A tampon according to claim 4 wherein the overwrap has a horizontal gravimetric wicking capacity of at least about 3 grams of fluid per gram of overwrap.

6. A tampon according to claim 3 wherein the overwrap has a horizontal gravimetric wicking capacity of at least about 1 gram of fluid per gram of overwrap.

7. A tampon according to claim 1 wherein the overwrap has a horizontal gravimetric wicking capacity of from about 6 to about 10 grams of fluid per gram of overwrap.

8. A tampon according to claim 1 wherein the overwrap has a horizontal gravimetric wicking capacity of from about 6 to about 10 grams of fluid per gram of overwrap.

9. A tampon according to claim 1 wherein the fluid wicking overwrap has a low adherence to tissue.

10. A tampon according to claim 1 wherein the overwrap comprises about 50% polypropylene and about 50% rayon.

11. A tampon according to claim 10 wherein the overwrap is thermally bonded layered.

12. A tampon according to claim 10 wherein the overwrap is mechanically altered by a method selected from the group consisting of ring-rolling or aperaturing.

13. A tampon according to claim 1 wherein the overwrap comprises about 50% rayon and about 50% polyester.

14. A tampon according to claim 13 wherein the overwrap is hydroentangled and layered.

15. A tampon according to claim 13 wherein the overwrap is mechanically altered by a method selected from the group consisting of ring-rolling or aperaturing.

16. A tampon according to claim 1 wherein the generally elongated absorbent member has an insertion end and a withdrawal end and wherein the overwrap extends at least about 2 mm beyond the withdrawal end of the generally elongated absorbent member to form a skirt.

17. A tampon according to claim 1 wherein the overwrap is resin bonded.

18. A tampon according to claim 1 wherein the synthetic fibers are selected from a group consisting of polyester, polyolefin, nylon, or mixtures thereof.

19. A tampon according to claim 1 wherein the synthetic fibers are selected from a group consisting of polypropylene, polyethylene, polyacrylic, cellulose acetate, or mixtures thereof.

20. A tampon according to claim 1 wherein the synthetic fibers comprise bicomponent fibers.
